(19) United States
(12) Patent Application Publication
(10) Pub. No.: US 2017/0007126 A1
(43) Pub. Date: Jan. 12, 2017

(54) SYSTEM FOR CONDUCTING A REMOTE PHYSICAL EXAMINATION

(71) Applicant: DOC@HOME LTD, RAMAT GAN (IL)

(72) Inventor: Amir SHAHAR, RAMAT GAN (IL)

(73) Assignee: DOC@HOME LTD, RAMAT GAN (IL)

(21) Appl. No.: 15/272,450

(22) Filed: Sep. 22, 2016

Related U.S. Application Data
(63) Continuation-in-part of application No. PCT/IL2015/050294, filed on Mar. 19, 2015.
(60) Provisional application No. 61/969,180, filed on Mar. 23, 2014.

(51) Int. Cl. A61B 5/00; A61B 5/0402; A61B 5/08; A61B 5/117; A61B 5/1455; G06F 19/00; A61B 5/021; A61B 5/20; A61B 1/04; A61B 1/267; A61B 1/227; A61B 5/0205; A61B 5/145

(52) U.S. Cl. CPC A61B 5/0002; A61B 5/0022; A61B 5/02055; A61B 5/0402; A61B 5/08; A61B 5/117; A61B 5/14551; A61B 5/14532; A61B 5/021; A61B 5/20; A61B 1/04; A61B 1/267; A61B 1/227; G06F 19/3406; G06F 19/3418; G06F 19/345

(57) ABSTRACT

The present invention provides a portable holdable device, system and method for conducting a remote primary medical examination, the device comprising: at least one input means for collecting self generated measurements of at least one health characteristic of a patient; a wireless network connection for receiving and transferring at least one signal from the input means to a physician thereby, transferring physiological data of the patient. The wireless network connection communicates with a user interface platform (UIP); the UIP comprises a predetermined protocol comprising a plurality of medical characteristics and conditions selectable according to patient's current physical condition. The self generated measurements of the patient are collectable, storable and deliverable by said input means on demand by said physician.

### CROSS REFERENCE TO RELATED APPLICATIONS

This application is a Continuation in Part of International PCT Application Number PCT/IL2015/050294, filed on Mar. 19, 2015, which claims priority from U.S. Provisional Application No.: 61/969,180, filed on Mar. 23, 2014. Both of these applications are hereby incorporated by reference in their entirety.

### FIELD OF THE INVENTION

The present invention pertains to a device and system for conducting a remote physical examination and further diagnosis, more particular the present invention provides a system for conducting a remote physical examination according to a user interface platform (UIP) comprising a predetermined protocol.

### BACKGROUND OF THE INVENTION

The field of transferring medical information via technological means comprises variety of solutions enabling measuring and collecting physical data of a known patient, either by a several steps or as automatically and further delivers the collected physical data to a computerized or human operated center.

One such known technology is a system for monitoring chronic patients consisting of measuring equipment attached to a telephone or to a telephone line, and transfer results of an ECG monitor, blood sugar level or other data to a computerized or human operated center.

There is also a known tool for transmitting medical data regarding a patient, including free text questions and answers, all are received filed and kept in a patient digital file.

An additional system comprises predetermined medical values classified as urgent and result in the transfer of the data to a physician or other qualified personnel. This system classify only events that are classified in advance are as urgent and then brought to the attention of the medical staff, while all other patient related data is stored in the digital file.

US patent app. No. 200102932 provides a system for computerized, knowledge-based medical diagnostic and treatment advice. The medical advice is provided to the general public over networks, such as a telephone network or a computer network. The invention also includes a stand-alone embodiment that may utilize occasional connectivity to a central computer by use of a network, such as the Internet. Two new authoring languages, interactive voice response and speech recognition are used to enable expert and general practitioner knowledge to be encoded for access by the public. “Meta” functions for time-density analysis of a number of factors regarding the number of medical complaints per unit of time are an integral part of the system. A re-enter feature monitors the user's changing condition over time. A symptom severity analysis helps to respond to the changing conditions; System sensitivity factors may be changed at a global level or other levels to adjust the system advice as necessary.

US patent app. No. 20070156626 discloses a system and method of remote patient monitoring to allow a patient to initiate and activate sensing systems. In the system and method, standard parameters can be sensed, and the information can then be processed and sent to the physician or clinician. The clinician then has the ability to remotely configure or reconfigure the parameters of the sensing system so as to probe for more targeted information based on the initial sensed data.

The known remote monitoring systems have been designed for patients with chronic diseases. Therefore, there is a long unmet need for a device having targeted applications accessibility and applicability to all the standard population and further reduces inequality of health care services between the population. Furthermore, there is a need for a device and system which will define and perform a full patient physician appointment, whilst it's not restricted to a predefined place or time frame and further provides a quality healthcare treatment and service.

### SUMMARY OF THE INVENTION

It is hence another object of the invention to provide a system for conducting a remote primary medical examination, comprising:

(a) a portable remote device for performing a self medical examination, comprising:

(i) at least one input means for self generated measurements of at least one health characteristic of the patient; and (ii) a connection to a wireless network for receiving and transferring at least one signal from the input means to a physician thereby, transferring physical data of the patient;

(b) a user interface platform (UIP) for communicating with at least one selected from the group consisting of a portable device, a patient, a physician and a combination thereof; (c) at least one processor for processing data of the self generated measurements; and, (d) a memory unit for storing said self-generated measurement; (e) at least one communication unit for transferring and/or receiving the self generated measurements, a main server preprogrammed for data exchange with said at least one communication unit and interconnecting said patient and a health care personnel. The wireless network connection communicates with the UIP; the UIP comprises a predetermined protocol comprising a a plurality of medical characteristics and conditions selectable in real time according to patient's current physical condition; the self generated measurements are collectable, storable and deliverable by the input means on demand by the physician; further wherein the portable remote device transfers the self generated measurements data to the communication unit and the processor via the wireless connection such that the physician receives the self generated measurements according to an emergency classification predefined within the UIP.

It is a core purpose of the present invention to provide said processor preprogrammed for validating obtained self-generated measurements on generation thereof in real time.

It is within the scope of the invention to provide said processor provided with feature vectors defining self-generated measurements to be validated.

It is within the scope of the invention to provide said memory unit configured for storing said self-generated measurements for establishing an acceptable normal range by a machine learning procedure.

It is within the scope of the invention to provide said main server comprising a storage unit configured for storing medical records.

It is within the scope of the invention to provide said server preprogrammed for big data analyzing said obtained self-generated measurements; big data analysis comprises selecting essential features and predictive modelling base of on similar medical records.

It is within the scope of the invention to provide said portable device configured for providing said patient with physician's instructions in real time.

It is within the scope of the invention to provide said processor is configured for carrying out off-line self-generated measurements and transmitting results thereof to said main server.

It is within the scope of the invention to provide the aforementioned system, wherein the UIP stores the measurements data and further physiological data of the patient.

It is within the scope of the invention to provide the aforementioned system, wherein the self generated measurements comprising at least one data source selected from the group consisting of physiological data, physical data, medical history data, text messages and a combination thereof.

It is within the scope of the invention to provide the aforementioned system, wherein the UIP comprising a ranking mechanism for ranking patient's and the measurements data according to at least one parameter selected from the group consisting of patient's age, gender, race, emotional condition, physical condition, health condition and combination thereof.

It is within the scope of the invention to provide the aforementioned system, wherein the processor comprises a server with a set of operations executed by at least one processor, the operations are:

(a) receiving self generated measurements data of a patient from the portable device;

(b) analyzing the self generated measurements data;

(c) processing the self generated measurements data;

(d) determining the emergency classification of the self generated measurements; and

(e) transferring the self generated measurements data to the physician;

It is within the scope of the invention to provide the aforementioned system, wherein the processor comprises a patient and a physician registration page.

It is within the scope of the invention to provide the aforementioned system, wherein the device additionally comprising a USB exit.

It is within the scope of the invention to provide the aforementioned system, wherein the processor comprises a personal medical file of each patient medical history, current medical data, allergies, chronic and medical treatment.

It is within the scope of the invention to provide the aforementioned system, wherein the UPI further comprising a database for collecting the measurements.

It is within the scope of the invention to provide the aforementioned system, wherein the processor further comprising a database for collecting the measurements.

It is within the scope of the invention to provide the aforementioned system, wherein the processor further transmits an alert to the physician when the measurement is entered to the system.

It is within the scope of the invention to provide the aforementioned system, wherein the physiological data of the patient and the self generated measurements are selectable by the physician.

It is within the scope of the invention to provide the aforementioned system, wherein the portable device is configured for physical examination of at least one body part selected from the group consisting of: eyes, ears, nose, mouth cavity and throat, respiratory system, skin heart, abdomen and any combination thereof.

It is within the scope of the invention to provide the aforementioned system, wherein the portable device is configured for physical examination of at least one physical parameter selected from the group consisting of: blood pressure, heart rate, temperature, glucose level, blood-oxygen level, urine, bloodwork and any combination thereof.

It is within the scope of the invention to provide the aforementioned system, wherein the measurements data is send to the physician combined with patient's identification details.

It is within the scope of the invention to provide the aforementioned system, wherein the portable device is configured to perform a physical examination of at least one physical symptoms selected from the group consisting of pain of any organ, fever, chills, infection, weakness, fainting, syncope, palpitations, dizziness, instability, nausea, vomiting, diarrhea, constipation, rash, pruritus, itching, swelling ,lump, contusion, trauma, frequency/urgency (urine, stool), cough, dyspnea, wheezing, shortness of breath, bleeding, tiredness, confusion, restlessness, tremor, edema, blurred vision, wound, fall, heartburn, burn, plegia, allergy and a combination thereof.

It is within the scope of the invention to provide the aforementioned system, wherein the input means is selected from the group consisting of least one sensor, camera, audio, microphone, voice record, visual display, thermometer, stethoscope, Electrocardiography (ECG), Otoscope, tongue depressor, blood pressure monitor, Pulse oximetry, Spirometer, Ultrasound,medical imaging, chemical test means, systems and techniques such as MRI, X-ray, C.T, NMR, and a combination thereof.

It is within the scope of the invention to provide the aforementioned system, wherein the sensor type is selected from the group consisting of temperature sensor, touch sensor, voice sensor, pulse sensor, motion sensor and a combination thereof.

It is within the scope of the invention to provide the aforementioned system, wherein the input means is activated by a single action selected from the group consisting of pressing, pushing, pulling, positioning, turning, sliding, triggering, sensing and any combination thereof.

It is within the scope of the invention to provide the aforementioned system, wherein the input means is activated by a single activation element selected from the group consisting of spring switch, pressing button, pushing button, pulling button, positioning button, turning button, sliding button, triggering button, sensing button and any combination thereof.

It is within the scope of the invention to provide the aforementioned system, wherein the system increases the health-care services quality and/or income ratio according to accessibility indicators.

It is within the scope of the invention to provide the aforementioned system, wherein the processor is adapted as a communicable device selected from the group consisting of a tablet, a kiosk, a CPU, a smart phone, a telephone and any combination thereof.

It is within the scope of the invention to provide the aforementioned system, wherein the system further schedules medical encounters between patient and a physician, using a predetermined rules and subject to instructions by both physician and the patient.

It is within the scope of the invention to provide the aforementioned system, wherein the system additionally comprising at least one algorithm selectively executed based on at least a portion of the received information.

It is within the scope of the invention to provide the aforementioned system, wherein the algorithm scores at least a portion of the received information and diagnoses a medical condition associated with the executed medical algorithm.

It is within the scope of the invention to provide a combined device for primary laryngoscopic and otoscopic examinations. The aforesaid device comprising: (a) an image sensor; (b) a first lens cooperatively disposed in front of said image sensor such that an image of a larynx of a patient is formed on said image sensor when said image sensor and said lens are inserted into a mouth cavity of said patient; (c) at least one source of light configured for illuminating of said larynx, an acoustic duct and a drum membrane; (c) a speculum configured for translating said light into said acoustic duct.

It is the purpose of the present invention is to provide said device rearrangeable between a first arrangement for said laryngoscopic examination and a second arrangement for said otoscopic examination. The first arrangement comprises said image sensor, first lens and at least one source of light cooperatively insertable into said mouth cavity; in said second arrangement, said speculum is disposed before said at least one source of light such that said light is inserted into end butt face and propagates therewithin in a lightguide manner; said speculum is provided with a second lens configured cooperatively with said first lens for forming an image of the acoustic duct and a drum membrane.

It is hence another object of the invention to provide a method of conducting a remote primary medical examination comprising step of:

(a) providing a system for performing a self physical examination, comprising: a portable remote device for performing a self physical examination, comprising:

(i) at least one input means for self generated measurements of at least one health characteristic of the patient, (ii) a connection to a wireless network for receiving and transferring at least one signal from the input means to a physician thereby, transferring the self generated data, (iii) a user interface platform (UIP) for communicating with at least one selected from the group consisting of a portable device, a patient, a physician and a combination thereof, (iv) at least one processor for processing the measurement data, and (v) at least one communication unit for transferring and/or receiving the measurement;

(b) performing a self physical examination according to a predetermined protocol in the portable remote device;

(c) transferring the self generated measurements data to the communication unit and the processor via the wireless connection such that the physician receives the self generated measurements according to an emergency classification predefined within the processor.

It is within the scope of the invention to provide the aforementioned method, wherein method additionally comprising step of transferring the self generated measurements data on demand by the physician.

It is within the scope of the invention to provide the aforementioned method, wherein the method additionally comprising step of storing the self generated measurements.

It is within the scope of the invention to provide the aforementioned method, wherein the step of providing the processor comprising server with a set of operations executed by at least one processor, the operations are:

(a) receiving self generated measurements data of a patient from the portable device;

(b) analyzing the self generated measurements data;

(c) processing the self generated measurements data;

(d) determining the emergency classification of the self generated measurements; and

(e) transferring the self generated measurements data to the physician;

It is within the scope of the invention to provide the aforementioned method, wherein the step of providing the processor comprising a personal medical file of each patient medical history, current medical data , allergies, chronic and medical treatment.

It is within the scope of the invention to provide the aforementioned method, wherein the step of providing the system comprising a database for collecting the measurement data.

It is within the scope of the invention to provide the aforementioned method, wherein the method additionally comprising step of transmitting an alert to the physician when the measurement is entered to the system.

It is within the scope of the invention to provide the aforementioned method, wherein the step of providing the portable device configured for physical examination of at least one body part selected from the group consisting of: eyes, ears, nose, mouth cavity and throat, respiratory system, skin, heart, abdomen and any combination thereof.

It is within the scope of the invention to provide the aforementioned method, wherein the step of providing the portable device configured for physical examination of at least one physical parameter selected from the group consisting of: blood pressure, heart rate, temperature, glucose level, blood-oxygen level, bloodwork, urine and any combination thereof.

It is within the scope of the invention to provide the aforementioned method, wherein the step of transferring the measurement to the physician combined with patient's identification details.

It is within the scope of the invention to provide the aforementioned method, wherein the step of providing the portable device configured to perform a physical examination of at least one physical symptoms selected from the group consisting of pain of any organ, fever, chills, infection, weakness, fainting, syncope, palpitations, dizziness, instability, nausea, vomiting, diarrhea, constipation, rash, pruritus, itching, swelling .lump, contusion, trauma, frequency/urgency (urine, stool), cough, dyspnea, wheezing, shortness of breath, bleeding, tiredness, confusion, restlessness, tremor, edema, blurred vision, wound, fall, heartburn, burn, plegia, allergy and a combination thereof.

It is within the scope of the invention to provide the aforementioned method, wherein the step of providing the portable device comprising input means selected from the group consisting of least one sensor, camera, audio, microphone, voice record, visual display, thermometer, stethoscope, Electrocardiography (ECG), Otoscope, tongue depressor, blood pressure monitor, Pulse oximetry, Spirometer and a combination thereof.

It is within the scope of the invention to provide the aforementioned method, wherein the step of providing the system comprising least one algorithm selectively executed based on at least a portion of the received information,

It is within the scope of the invention to provide the aforementioned method, wherein the algorithm scores at least a portion of the received information and diagnoses a medical condition associated with the executed medical algorithm.

### BRIEF DESCRIPTION OF THE DRAWINGS

In order to understand the invention and to see how it may be implemented in practice, a few preferred embodiments will now be described, by way of non-limiting example only, with reference to be accompanying drawings, in which:

FIGS. 1-2 illustrate the portable device of the present invention for conducting a remote primary self medical examination comprising a camera as input means;

FIGS. 3-4 illustrate the portable device of the present invention for conducting a remote primary self medical examination comprising a stethoscope as input means;

FIGS. 5-6 illustrate the portable device of the present invention for conducting a remote primary self medical examination comprising a ECG architecture as input means;

FIG. 7 illustrates a scheme of a system for conducting a remote primary self medical examination, of the present invention;

FIG. 8 illustrates a block diagram of the procedure for conducting a physician's appointment not restricted to a specific location and time.

FIGS. 9a and 9b illustrate schematic views of a combined device for primary laryngoscopic and otoscopic examinations

### DETAILED DESCRIPTION OF THE INVENTION

The following description is provided so as to enable any person skilled in the art to make use of the invention and sets forth the best modes contemplated by the inventor of carrying out this invention. Various modifications, however, will remain apparent to those skilled in the art, since the generic principles of the present invention have been defined specifically to provide product and method of the invention described herein.

The present invention provides a device, system and method for remotely examining patient healthcare characteristics and condition. Thereby, the present invention further provides a virtual appointment check-up device and system, allowing a patient or another subject which is not a physician to perform a primary self-physical examination and further a remote diagnosis according to instructions and guidance provided by a user interface platform (UIP)such as graphic user interface (GUI) in a smart phone. The self physical examination comprises variety of physical measurements, photo images, video images, voice records, patient's medical history data, written messages and combination thereof.

The physician does not have to be online when the physical measurements are conducted and further sent via the UIP. The system may comprise a plurality of sensor which further allows the patient to perform a personalized and accurate medical measurement at home, at work or on the go such that the physician can provide an accurate diagnosis respectively. The results of the examination may be sent to a server cloud. When the server receives an input, an alert, SMS or email request is sent automatically to the patient's physician, selected from a predefined physician and specialist list. The physician receiving the input is able to review, download and further examine incoming data using a user interface. The user interface may further allow the physician to examine and analyze incoming measurements data and medical results such as stethoscope recorded sounds, ECG, blood pressure, Oxygen saturation (S_{P}O₂₎, temperature values and further video and photo images. The physician may further record and/or send the diagnosis and further treatment and/or call the patient or conduct a video phonecall for prescription and/or further discussion, treatments and/or additional tests. The patient medical data is further searchable by the physician.

In accordance to the present invention, the system provides a physician located remotely from a patient to analysis and to follow-up patient's current medical condition and further delivering diagnosis and further treatment accordingly.

In accordance to the present invention, the system provides a patient located remotely from medical services, a quality of care equal to patients living near medical services.

In another embodiment of the present invention, the portable device comprises input means for measuring and indicating patient's current medical condition. The input means is a probe end of the device, selected from the group consisting of at least one sensor, camera, audio, microphone, voice record, visual display, thermometer, stethoscope, Electrocardiography (ECG), Otoscope, tongue depressor, blood pressure monitor, Pulse oximetry, Spirometer and a combination thereof. The sensors may be based upon touch sensitive technology. The sensor type is selected from the group consisting of temperature sensor, touch sensor, voice sensor, pulse sensor, motion sensor and a combination thereof. The input means is activated by a single action selected from the group consisting of: pressing, pushing, pulling, positioning, turning, sliding, triggering, scanning sensing and any combination thereof. The single activation element is selected from the group consisting of spring switch, pressing button, finger press, biometric scanning pushing button, pulling button, positioning button, turning button, sliding button, triggering button, sensing button, RFID barcode and any combination thereof. The input means can further be detached from the device and replaced with another input means to perform an additional examination

In another embodiment of the present invention, the input means transfer an electronic signal to a user interface platform (UIP) via the wireless connection such as a Bluetooth connection, antenna, wireless USB, wireless sensor networks, satellite communications, mobile data service, ibeacon, zigbee, GPS and a combination thereof for receiving the medical measurement data examined by the portable device according to instructions provided by the UIP.

In another embodiment of the present invention, the UIP may be based upon a step-by-step procedure such as an algorithm and/or a cloud service.

In another embodiment of the present invention, the portable device is configured for physical examination of at least one body part selected from the group consisting of: eyes, ears, nose, mouth cavity and throat, respiratory system, skin, heart, abdomen and any combination thereof. In another embodiment of the present invention, the portable device is configured for medical examination of at least one physical parameter selected from the group consisting of: blood pressure, heart rate, temperature, glucose level, blood-oxygen level, bloodwork, urine and any combination thereof. The portable device is further configured to perform a medical examination of at least one physical symptoms selected from the group consisting of pain of any organ, fever, chills, infection, weakness, fainting, syncope, palpitations, dizziness, instability, nausea, vomiting, diarrhea, constipation, rash, pruritus, itching, swelling (glands, skin, soft tissue, joint, bone),lump, contusion, trauma, frequency/urgency (urine, stool), cough, dyspnea, wheezing, shortness of breath, bleeding (from any organ),tiredness, confusion, restlessness, tremor, edema, blurred vision, wound, fall, heartburn, burn, plegia, allergy and a combination thereof.

In another embodiment of the present invention, the measurements taking by the patient or another subject can be further viewed and heard online and real time by the patient or any other subject upon the screen of the portable device or a processor such as a smart phone or the computer screen. The system may further provide an approval or verification such as ‘OK’ message that the measurements data was transferred successfully to the physician and/or the medical heath institute. The verification may be provided by a message format, an image format or an alert such as a designated sound sent by the UIP.

In another embodiment of the present invention, the portable device for conducting remote physical measurements may comprise a touchpad, or can further be controlled using “voice actions”. To activate the device, the user may say “O.K.” Once the device is activated, the user can say an action, such as “Take a picture”, “Record a video”, and the like

In another embodiment of the present invention, the system for conducting a full patient-physician medical encounter initiated by a patient or a physician is based upon at least one of the following processes verification, certification, authentication and validation. Each of these processes may be a sub process of the other or a independent process of the present invention system. The level of verification, or number of times the producer's data is verified against is not limited and relay upon the user's actions and inputs. In another embodiment of the present invention, the system may be based upon intuitive physical examination of the patient or another subject which is not a physician.

The term ‘medical examination’ herein refers to a physical examination, medical analysis or clinical examination (more popularly known as a check-up or medical) therefor, the process by which a medical professional investigates the body of a patient for signs of disease. It generally follows the taking of the medical history—an account of the symptoms as experienced by the patient. Together with the medical history, the physical examination aids in determining the correct diagnosis and devising the treatment plan. This data then becomes part of the medical record. The physical examination may further include evaluation of general patient appearance and specific organ systems and further vital signs of temperature examination, pulse and blood pressure.

The term ‘self examination’ herein refers to physical examination conducted by a patient itself or by another subject which is not a physician.

The term ‘self generated measurements’ herein refers to physical measurements or medical check up conducted by a patient itself or by another subject which is not a physician.

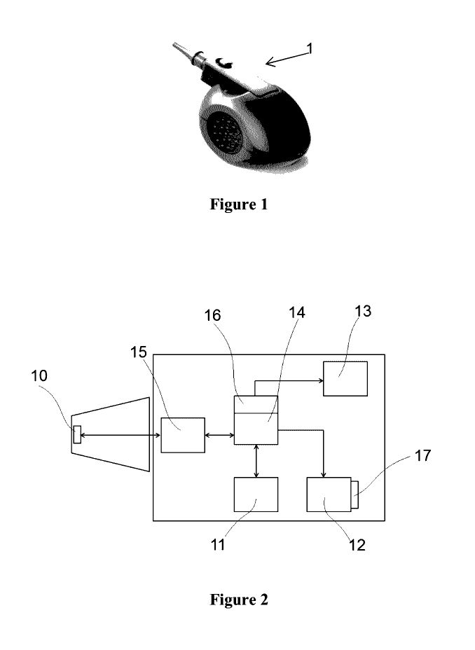

Reference is now made to FIGS. 1 and 2 which illustrate a holdable portable remote device adapted for performing a remote self medical examination by a patient or another subject which is not a physician. The portable remote device comprising: (i) at least one video input means 10 such as a CMOS array for self generated measurements optimally in real time of at least one health or medical characteristic of the patient and, (ii)a wireless network 12 provided with antenna 17 for receiving and transferring at least one signal from the input means to a physician thereby transferring measurement physiological and physical data of the patient on demand by a physician.

An electric signal from CMOS array 10 via analog-digital converter 15 is processed in processor unit 14. Numeral 16 refers to memory unit storing the obtained self-generated measurements. The system is energized by power circuit 11.

In another embodiment of the present invention, the patient may further provide a written or voice description, photo or any image of his medical condition and symptoms to the user interface platform (UIP) for receiving a self medical examination procedure accordingly and further to be sent to the physician.

The wireless network connection 12 communicates with the UIP which comprises a predetermined protocol comprising a plurality of medical characteristics and/or conditions such that a patient may select a relevant characteristic and/or condition from a list according to patient's current physical condition. Thereby, the patient performs a self generated measurements using the input means respectively.

In another embodiment of the present invention, FIG. 2 further presents a camera probe end 10 as input means which can be replaced detached and/or removed according to the predefined diagnosis and patient condition respectively.

In another embodiment of the present invention, the device additional comprises a power supply unit 11 which may include any suitable portable power supply unit, e.g., a battery, a rechargeable battery, and the like. The device further comprises a USB exit 13 for additional connection, communication, and/or power supply between CPU and additional electronic devices. Furthermore the portable device is a handheld device allowing online communication and further recording of data when not online

In another embodiment of the present invention, the portable device is programmed with software instructions configured to interact with the patient to deduce a plurality of possible medical conditions. The deduced list of possible medical conditions is provided to the patient according to the patient's description of his current medical condition. Suggested diagnosis and/or treatment plans may also be provided based on the list of possible medical conditions.

The processor 14 is preprogrammed for validating obtained self-generated measurements on generation thereof in real time. The processor 14 provided with feature vectors defining self-generated measurements to be validated. The memory unit 16 is configured for storing said self-generated measurements for establishing an acceptable normal range by a machine learning procedure. The main server (not shown) comprises a storage unit configured for storing medical records. The server preprogrammed for big data analyzing said obtained self-generated measurements; big data analysis comprises selecting essential features and predictive modelling base of on similar medical records.

In another embodiment of the present invention, the portable device further comprises processor unit 14 as a mathematical manipulation of an information signal and further to modify or improve it. The DSP is characterized by the representation of discrete time, discrete frequency or other discrete domain signals by a sequence of numbers or symbols and the processing of these signals. The DSP measures filter and/or compress continuous real-world analog signals. The first step comprises converting the signal from an analog to a digital form, by sampling and then digitizing it using an analog-to-digital converter 15 (ADC), which turns the analog signal into a stream of numbers. However, the required output signal may be another analog output signal, which requires a digital-to-analog converter (DAC).

In another embodiment the portable device of the present invention allows a patient to perform an annual physical examination is a source of reassurance that they're as healthy as they feel or as an alarm system, to catch health problems before they become serious.

The system may further provide questions about important behaviors, like smoking, excessive alcohol use, sexual health, diet, and exercise, The device can check on patient vital signs, vaccination status and further update and store patient personal and family medical history. The vital sign which the portable device can examine are for example: blood pressure: less than 120 over 80 is a normal blood pressure. The device can be defined a high blood pressure (hypertension) is 140 over 90 or higher and further may provide an alert to the patient and his physician when the measurements result is outside the defined range and limit

Heart rate: Values between 60 and 100 are considered normal. Many healthy people have heart rates slower than 60, however.

Respiration rate: Around 16 is normal. Breathing more than 20 times per minute can suggest heart or lung problems.

Temperature: 98.6 degrees Fahrenheit is the average, but healthy people can have resting temperatures slightly higher or lower.

The device may further conduct an online conversation of patient-physician. By performing an additional patient-physician conversation the physician gathers additional information about the patient and health just by watching and talking to the patient. For example the physician may further ask questions such as, How is your memory and mental quickness? Does your skin appear healthy? Can you easily stand and walk?.

The device can further listen to patient heart with the input means of the device such as a stethoscope, a doctor might detect an irregular heartbeat, a heart murmur, or other clues to heart disease following the measurements data sent to him Furthermore, using a stethoscope, a device can listens for crackles, wheezes, or decreased breath sounds. These and other sounds are clues to the presence of heart or lung disease.

Opening up and saying “ah” shows off the patient throat and tonsils. The quality of patient teeth and gums also provides information about patient overall health. Ears, nose, sinuses, eyes, lymph nodes, thyroid, and carotid arteries may also be examined

The device is with the ability to use a range of examination techniques including tapping patient abdomen to detect liver size and presence of abdominal fluid, listening for bowel sounds with a stethoscope, and palpating for tenderness. Nerves, muscle strength, reflexes, balance, and mental state may be assessed.

Dermatological Exam, skin and nail findings could indicate a dermatological problem or disease somewhere else in the body.

Extremities Exam, the device may further measure physical and sensory changes. Pulses can be checked in patient arms and legs. Examining joints can assess for abnormalities.

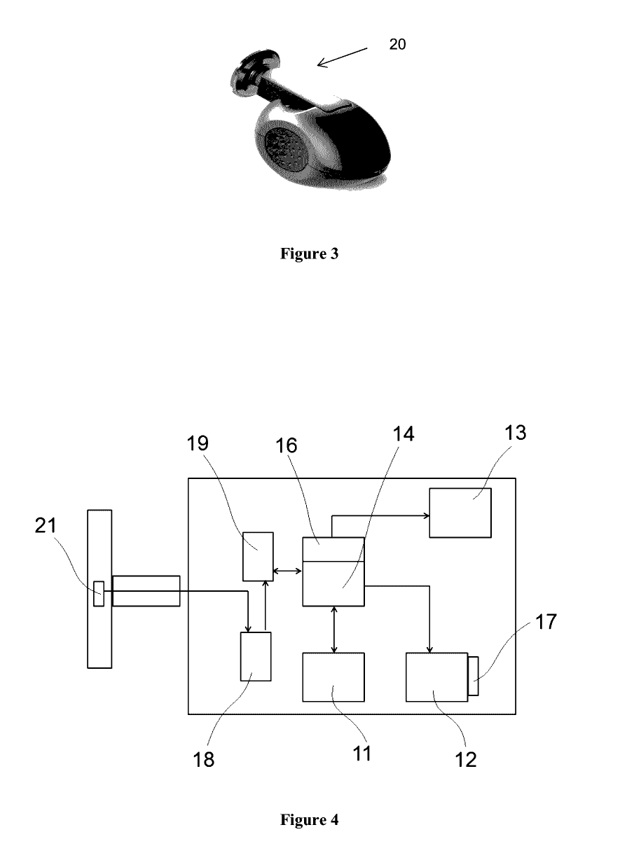

Reference is now made to FIGS. 3 and 4 which illustrate a holdable portable remote device adapted for performing a remote self medical examination by the patient or a subject which is not a physician. The portable remote device comprising a stethoscope probe end as input means 20 for self generated measurements of at least one health or medical characteristic of the patient. The stethoscope is used for auscultation, or listening to the internal sounds of patient's lung and heart sounds. It is also used may be used for auscultation intestines and blood flow in arteries and veins.

In another embodiment of the present invention, the stethoscope comprises connection pins for device engagement, spring switch trigger 26 for activating the device and further recording and storing the measurements results. In accordance with the present invention, the input means may further comprises microphone 21, amplifier and filter circuits 18 for low frequencies and noise reduction.

In another embodiment of the present invention, the input means may further comprise at least one sensor for sensing variety of physiological characteristics.

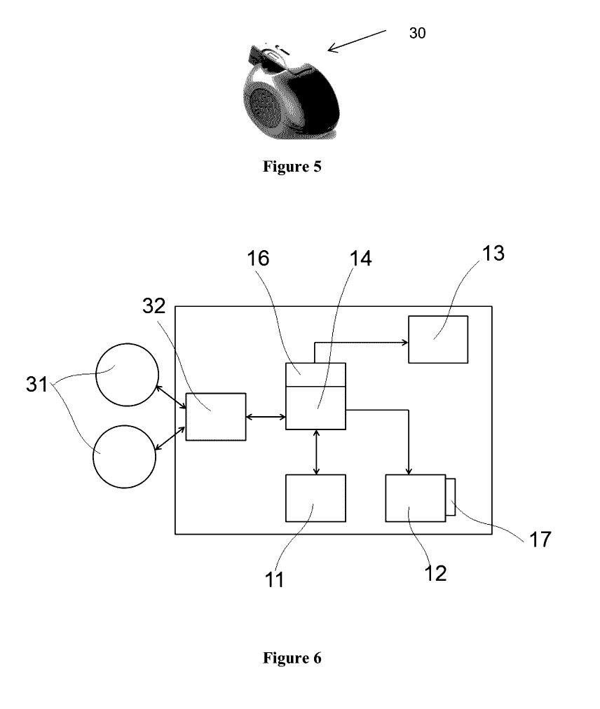

Reference is now made to FIGS. 5 and 6 which illustrate a holdable portable remote device adapted for performing a remote self medical examination by a patient or a subject which is not a physician. The portable remote device comprising a ECG (e.g EKG) 30 probe end as an input means for measuring the heart's electrical conduction system of a patient. The ECG input means comprises a finger touch sensors 31. The ECG input means can further be detached from the device and replaced with another input means to perform an additional examination. The ECG is a replaceable probe which may be moved axially along the rotation axis for alignment with an alignment axis. Preferably, the head is moved axially into engagement for coupling and engagement therewith in a substantially sandwiched manner

In another embodiment of the present invention, the device may further include a glass which are designated for viewing the measurements data only by the glass wearer. The glasses may comprise an optical head-mounted display (OHMD). The Glass displays information may be in a smartphone-like hands-free format, which can communicate with the Internet via natural language voice commands.

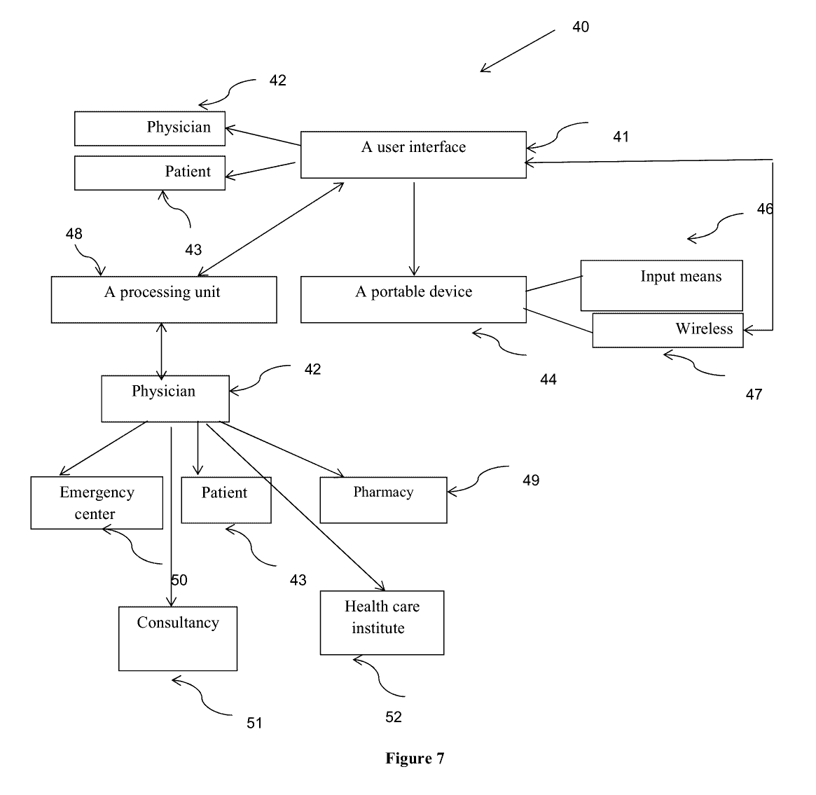

Reference is now made to FIG. 7 which presents a scheme of a system for conducting a remote primary medical examination not restricted to a specific location and time 40, comprising:

(a) a portable remote device 44 for performing a self medical examination, comprising:

(i) at least one input means 46 for self generated measurements of at least one health or medical characteristic of the patient; the input means may be a removable and replicable probe end; and (ii) a connection to a wireless network 47 for receiving and transferring at least one signal from the input means to a physician thereby, transferring self generated measurements data,

(b) a user interface platform (UIP) 41 for communicating with at least one selected from the group consisting of a portable device, a patient, a physician and a combination thereof,

(c) at least one processor unit 45 for processing the self generated measurements data and,

(d) at least one communication unit 48 for transferring and/or receiving the self generated measurements. The UIP 41 comprises a predetermined protocol comprising a plurality of selectively medical characteristics and/or conditions such that the patient 43 selects a relevant characteristic and/or condition from a list according to patient's current physical condition thereby, the patient perform the self medical examination using the portable device respectively. Furthermore, the portable remote device 44 transfers the measurement data to the communication unit 48 and the processor 45 via the wireless connection 47 such that the physician receives the self generated measurements according to an emergency classification predefined within the UIP 41.

In another embodiment of the present invention, the physician may receive automatically patient's measurements data and results, on demand or by selecting patient's measurements and medical data. The physician analysis and examine the delivered measurement data and further provides a diagnosis accordingly and may decide to transfer the diagnosis according to the urgency or emergency of the patient status. The physician's diagnosis is transferred to at least one of the following: the patient 43, the emergency center 50 ,the pharmacy 49, an additional physician or consultancy 51, or/and a healthcare institute 52 which are connected to the system.

In another embodiment of the present invention, the patient may further provide a written or voice description, photo or any image of his medical condition and symptoms within the user interface for receiving a self medical examination procedure accordingly and further to be sent to the physician.

In another embodiment of the present invention, the processor may comprise a personal medical file of each patient medical history, current medical data, allergies, chronic and medical treatment. Furthermore the processor further transmits an alert to the physician when the self generated measurements are entered to the system.

In another embodiment of the present invention, the system comprises a registration page for both the patient and the physician. The registration page comprises the patient and physician identification information.

In another embodiment of the present invention, the system is further synchronized with patient medical history, stored in the processor unit.

The processor may be a server having data base for archive all patient registration information and medical history retrieved by hospitals, medical clinics and/or physician.

In another embodiment of the present invention, the processor is adapted as a communicable device selected from the group consisting of a tablet, a kiosk, a CPU, a smart phone, a telephone and any combination thereof.

In another embodiment of the present invention, the patient registers into the server. Upon registering to the service the patient creates a personal medical file, with basic data of past medical history, current medical problems, general allergies, drug allergy, chronic medical treatment and drugs and the like.

In another embodiment of the present invention, the system provides a disease-specific guidance subjects, users and/or patients to enable accurate diagnosis and treatment respectively. The system may further provide and based the analysis upon knowledge of specific diseases, chronic patient or condition, acute patient or condition and patient history stored and/or downloaded to the CPU.

In another embodiment of the present invention, the system, according to each condition provided by the patient, provides instructions for home-self physical examination concerning heart auscultation, lungs, abdomen, inspection of the ear canal, ear drum, inspection of the mouth cavity and throat, inspection of the skin, measuring heart rate, performing an ECG, oximetry measurements, perfuming spirometry, measuring body parts and the like. The home-self physical examination is transferred to the physician via the user interface platform and the processor unit for further analysis. The physical examination may be further performed at the patient or subject home or any other location or place.

In another embodiment of the present invention, the system is based upon a step-by-step procedure for calculations such as an algorithm and/or a cloud service.

The system of the present invention provides a quality improvement of a primary medical care without using a physician. The system further provides accessibility optimize their effectiveness in quality improvement strategies.

The system further comprises an emergency classification which further grades the urgency of the received measurements data. Furthermore, the physician will receive an alert when a real time examination massage is entered system. According to data received, the physician provides a diagnosis and further instructions to the patient, a medical center and/or a pharmacy.

In another embodiment of the present invention, the system further allows the physician to contact the patient via the user interface platform or any other electronic means for further information or updates. The system may further communicate with additional network resources such as hospitals, health maintenance, organization (HMO) and the like which further stores patient medical records history. The system may further comprise a plurality of medical measurements results having classified ranges and parameters according to normal range of healthy people in a certain group depending upon patient's age, gender, race, emotional condition, physical condition, health or medical condition and other medical known factors and further abnormal results which fall outside the normal range.

In another embodiment of the present invention, the system further allows ranking and prioritizing a patient according to at least one of the following: patient's age, gender, race, emotional condition, symptom, a group of symptoms, physical condition, health or medical condition and combination thereof. The ranking mechanism further allows the physician to select and prioritized a patient in the system and further perform a medical analysis following patients measurements data stored in the system.

In another experimental embodiment, if the system receives an incoming physical measurement data of a patient which is classified as old, depressed and sick the processor will rank the patient as a high priority patient and will send to the physician an alert or worming via the UIP.

The system of the present invention further provide increment of the healthcare-related quality of life by using indicators which are explicitly defined and measurable items referring to the structures, processes, or outcomes of medical care. Indicators are operationalized by using review criteria and standards and further according to local context and patient circumstances. The system is based upon consensus techniques which further facilitate quality improvement by allowing a broader range of aspects of care to be assessed and improved. The indicators are used within quality improvement approaches that focus on whole healthcare systems

In another embodiment of the present invention, the system may further provide feedback to the patient and/or the physician according to the current condition of the patient, the physician diagnosis or the medical status and history of the patient.

In another embodiment of the present invention, the system further comprises algorithm and/or technical means for detecting, analyzing and improving images, sounds and vibration received and recorded by the portable device. The smart phone comprising a UIP will perform an automatic analysis of the data and measurements transferred by the portable device. For example input voices or sounds of a patient lungs recorded by the portable device, will further be analyzed to determine the type of the sound and acceptable sound quality, respectively. When the system will identify the type of the sounds as respiratory sound the sound will further be analyzed to detect the type of the patient breath sounds compering to a base line of a normal breath sounds. The algorithm may examine physical parameters such as sound pressure level, frequency, and modulation depth and correlate them to human hearing perception or with a known normal sound of the analyzed organ.

The system will classify the sound according to predefined sounds for example, Vesicular—similar to a soft rustle (of leaves). The inspiratory sounds are faster and louder and longer than expiratory sounds. The expiratory intensity is soft and the pitch is low die away after the first third of the expiratory phase.

Bronchovesicular—heard over the 1st and 2nd intercostal spaces and the interscapular area. The inspiratory and expiratory phases are roughly equal in length.

Bronchial—may be normally heard over the manubrium. Expiratory phase is greater than inspiratory. The expiratory pitch is high and intensity is loud.

Tracheal—heard directly over the trachea. Inspiratory phase equals the expiratory phase. The sound is very loud and the pitch very high.

The system may further comprise image processing means such as digital image processing, and analog image processing for improving the received image from the portable device measurement results. The image processing is any form of signal processing for which the input is an image, such as a photograph or video frame; the output of image processing may be either an image or a set of characteristics or parameters related to the image. The image-processing techniques involve treating the image as a two-dimensional signal and applying standard signal-processing techniques to it.

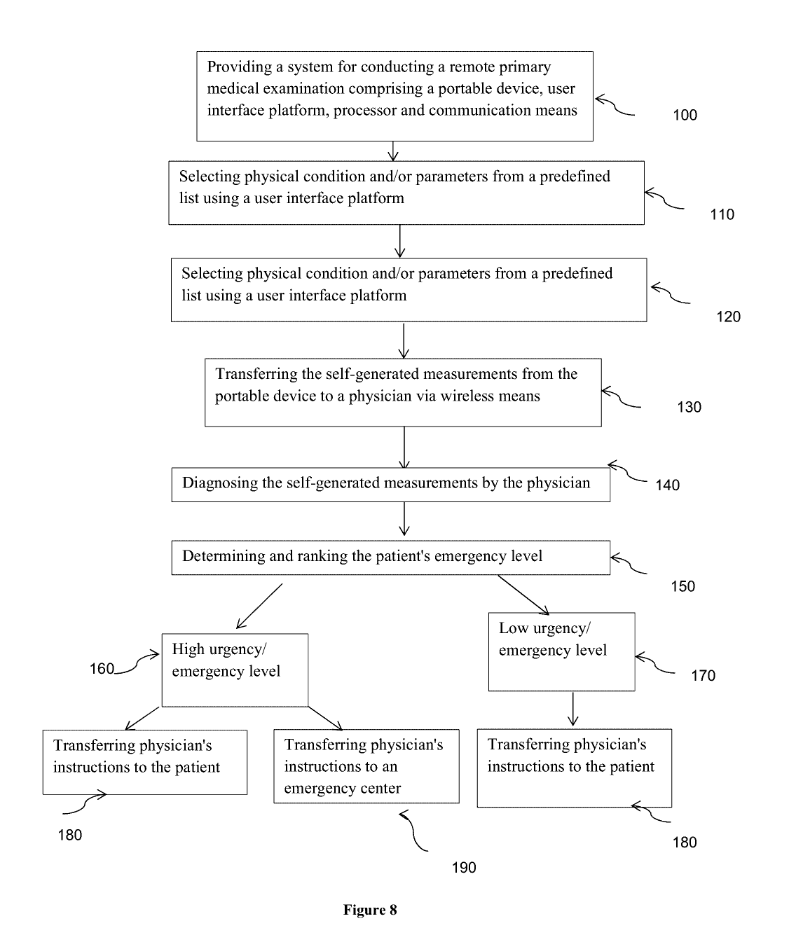

Reference is now made to FIG. 8 which illustrates a method of conducting a physician's appointment not restricted to a specific location and time, comprising step of: (a) providing a system for performing a self physical examination, comprising: (i) a portable remote device, comprising: at least one input means for self generated measurements of at least one health or medical characteristic of the patient; a connection to a wireless network for receiving and transferring at least one signal from the input means to a physician thereby, transferring the self generated measurements data on line or offline optionally,

(ii) a user interface platform (UIP) for communicating with at least one selected from the group consisting of a portable device, a patient, a physician and a combination thereof, (iii) at least one processor for processing the self generated measurements data; and

(iv) at least one communication unit for transferring and/or receiving the self generated measurements,

(b) performing a self physical examination according to a predetermined protocol in the portable remote device,

(c) transferring the self generated measurements data to the communication unit and the processor via the wireless connection such that the physician receives the self generated measurements according to an emergency classification predefined within the processor.

In another embodiment of the procedure for performing a self physical examination comprising step of: providing a system for conducting a remote primary medical examination,

selecting physical condition and/or parameters from a predefined list using a user interface platform (110), performing a physical examination using a portable device according to a predetermined protocol (120), transferring the physical examination results from the portable device to a physician via wireless means (130), diagnosing and analyzing the self-generated measurements by the physician and further evaluating the patient condition, disease or disorder accordingly (140), following the physician's diagnostic the physician is further determining the emergency level (150): high urgency/emergency level (160) or low urgency/emergency level (170).

If the emergency level is of high urgency/emergency level the physician's instructions are transferred to the patient and to an emergency center 190. If the emergency level is of low urgency/emergency level the physician's instructions are transferred to the patient 180 and/or pharmacy if needed.

In another embodiment of the present invention, the method additionally comprises the step of providing the processor comprising a personal medical file of each patient medical history, current medical data, allergies, chronic and medical treatment.

In another embodiment of the present invention, the method additionally comprises the step of transmitting an alert to the physician when the self generated measurements data is entered to the system.

In another embodiment of the present invention, the method additionally comprises the step of providing the portable device configured for physical examination of at least one body part selected from the group consisting of: eyes, ears, nose, mouth cavity and throat, respiratory system, skin, heart, gastrointestinal tract (GI) and any combination thereof.

In another embodiment of the present invention, the method additionally comprises the step of providing the portable device configured for physical examination of at least one physical parameter selected from the group consisting of: blood pressure, heart rate, temperature, glucose level, blood-oxygen level, bloodwork, urinalysis (UA), Spirometry and any combination thereof.

In another embodiment of the present invention, the method additionally comprises the step of transferring the self generated measurements to the physician combined with patient's identification details.

In another embodiment of the present invention, the method additionally comprises the step of providing the portable device configured to perform a physical examination of at least one physical symptoms selected from the group consisting of pain of any organ, fever, chills, infection, weakness, fainting, syncope, palpitations, dizziness, instability, nausea, vomiting, diarrhea, constipation, rash, pruritus, itching, swelling (glands, skin, soft tissue, joint, bone), lump, contusion, trauma, frequency/urgency (urine, stool), cough, dyspnea, wheezing, shortness of breath, bleeding (from any organ), tiredness, confusion, restlessness, tremor, edema, blurred vision, wound, fall, heartburn, burn, plegia, allergy and a combination thereof.

In another embodiment of the present invention, the method additionally comprises the step of providing the portable device comprising input means selected from the group consisting of least one sensor, camera, audio, microphone, voice record, voice input, visual display, thermometer, stethoscope, Electrocardiography (ECG), Otoscope, tongue depressor, blood pressure monitor, Pulse oximetry, Spirometer, Ultrasound, medical imaging, chemical test means, systems and techniques such as MRI, X-ray, C.T, NMR, and a combination thereof.

In another embodiment of the present invention the input means may comprises a chemical test means providing a qualitative or quantitative procedure designed to prove the existence of, or to quantify, a chemical compound or chemical group with the aid of a specific reagent in body tissue or fluids (e.g blood, fecal, urine or tears),

The input means may further comprise a separate wireless connection or a USB exit for further transmitting the measurements data to the physician or a health care institute. In another embodiment of the present invention, the method additionally comprises the step of providing the system comprising least one medical algorithm selectively executed based on at least a portion of the received information, wherein the medical algorithm scores at least a portion of the received information and diagnoses a medical condition associated with the executed medical algorithm if the score reaches or passes a threshold, wherein the diagnosed medical condition is communicated via the output device.

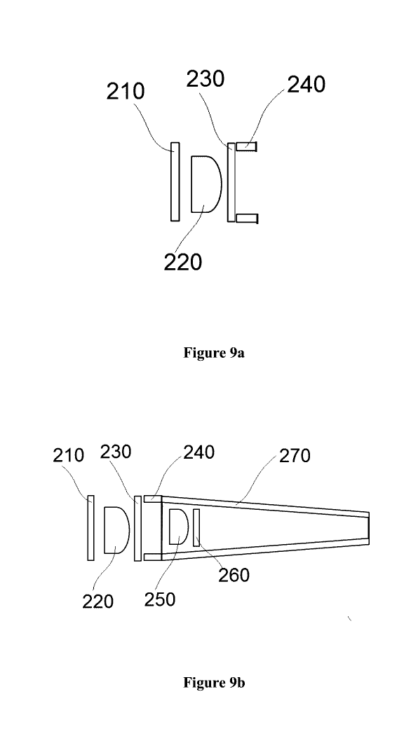

Reference is now made FIGS. 9a and 9b presenting a combined device for primary laryngoscopic and otoscopic examinations. In a first arrangement for laryngoscopic examination, the aforesaid device comprises: image sensor 210 and first lens 220 protected by a cover glass 230. The first lens is cooperatively disposed in front of the image sensor such that an image of a larynx of a patient when said image sensor and said lens are inserted into a mouth cavity of said patient. At least one source 240 of light is used for illuminating of said larynx, an acoustic duct and a drum membrane. In a second arrangement, speculum 270 is used for translating the light into the acoustic duct (not shown). In the device the present invention, speculum 270 is disposed before at least one source 240 of light such that the light is inserted into end butt face and propagates within speculum 270 in a lightguide manner The speculum 270 is provided with second lens 250 also protected by cover glass 260 which in cooperation with first lens forms an image of acoustic duct and a drum membrane. Disposable speculum 270 and second lens 250 are in the scope of the present invention.

The embodiments were chosen and described to provide the best illustration of the principals of the invention and its practical application, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. A system for conducting a remote primary medical examination, comprising:
a. a remote portable device for performing a medical self examination comprising:
i. at least one input means for self generated measurements of at least one health characteristic of said patient; and
ii. a connection to a wireless network for receiving and transferring at least one signal from said input means to a physician thereby, transferring physical data of said patient;
b. a user interface platform (UIP) for communicating with at least one selected from the group consisting of a portable device, a patient, a physician and a combination thereof;
c. at least one processor for processing data of said self generated measurements;
d. a memory unit for storing said self-generated measurement; and,
e. at least one communication unit for transferring and/or receiving said self generated measurements;
f. a main server preprogrammed for data exchange with said at least one communication unit and interconnecting said patient and a health care personnel;
said wireless network connection communicates with said UIP; said UIP comprises a predetermined protocol comprising a plurality of medical characteristics and conditions selectable in real time according to patient's current physical condition; said self generated measurements are collectable, storable and deliverable by said input means on demand by said physician; further wherein said portable remote device transfers said self generated measurements data to said communication unit and said processor via said wireless connection such that said physician receives said self generated measurements according to an emergency classification predefined within said UIP;
wherein said processor is preprogrammed for validating obtained self-generated measurements on generation thereof in real time.

2. The system according to claim 1, wherein at least one of the following is true:
a. said processor is provided with feature vectors defining self-generated measurements to be validated;
b. said processor is configured for carrying out off-line self-generated measurements and transmitting results thereof to said main server;
c. said memory unit is configured for storing said self-generated measurements for establishing an acceptable normal range by a machine learning procedure;
d. said main server comprises a storage unit configured for storing medical records;

3. The system according to claim 2, wherein said server is preprogrammed for big data analyzing said obtained self-generated measurements; big data analysis comprises selecting essential features and predictive modelling base of on similar medical records.

4. The system according to claim 1, wherein, wherein at least one of the following is true:
a. said self-generated measurements comprising at least one data source selected from the group consisting of physiological data, physical data, medical history data, text messages and a combination thereof;
b. said UIP comprising a ranking mechanism for ranking patient's and said measurements data according to at least one parameter selected from the group consisting of patient's age, gender, race, emotional condition, physical condition, health condition and combination thereof.

5. The system according to claim 1, wherein said server with a set of operations executed by at least one processor, said operations are:
a. receiving self generated measurements data of a patient from said portable device;
b. analyzing said self generated measurements data;
c. processing said self generated measurements data;
d. determining the emergency classification of said self generated measurements; and
e. transferring said self generated measurements data to said physician.

6. The system according to claim 1, wherein, wherein at least one of the following is true:
a. said processor comprises a patient and a physician registration page;
b. said device additionally comprising a USB exit;
c. said processor comprises a personal medical file of each patient medical history, current medical data, allergies, chronic and medical treatment.

7. The system according to claim 1, wherein at least one of the following is true:
a. said UPI further comprising a database for collecting said measurements;
b. said processor further comprising a database for collecting said measurements;
c. said processor further transmits an alert to said physician when said measurement is entered to said system;
d. said physiological data of said patient and said self generated measurements are selectable by said physician.

8. The system according to claim 1, wherein at least one of the following is true:
a. said portable device is configured for physical examination of at least one body part selected from the group consisting of: eyes, ears, nose, mouth cavity and throat, respiratory system, skin, heart, abdomen and any combination thereof;
b. said portable device is configured for physical examination of at least one physical parameter selected from the group consisting of: blood pressure, heart rate, temperature, glucose level, blood-oxygen level, bloodwork, urine and any combination thereof.

9. The system according to claim 1, wherein said measurements data is send to said physician combined with patient's identification details.

10. The system according to claim 1, wherein, wherein at least one of the following is true:
a. said portable device is configured to perform a physical examination of at least one physical symptoms selected from the group consisting of pain of any organ, fever, chills, infection, weakness, fainting, syncope, palpitations, dizziness, instability, nausea, vomiting, diarrhea, constipation, rash, pruritus, itching, swelling, lump, contusion, trauma, frequency/urgency (urine, stool), cough, dyspnea, wheezing, shortness of breath, bleeding, tiredness, confusion, restlessness, tremor, edema, blurred vision, wound, fall, heartburn, burn, plegia, allergy and a combination thereof;
b. said input means is selected from the group consisting of least one sensor, camera, audio, microphone, voice record, visual display, thermometer, stethoscope, Electrocardiography (ECG), Otoscope, tongue depressor, blood pressure monitor, Pulse oximetry, Spirometer, Ultrasound,medical imaging, chemical test means, systems and techniques such as MRI, X-ray, C.T, NMR, and a combination thereof;
c. said portable device is configured for providing said patient with physician's instructions in real time.

11. The system according to claim 1, wherein said sensor type is selected from the group consisting of temperature sensor, touch sensor, voice sensor, pulse sensor, motion sensor and a combination thereof.

12. The system according to claim 1, wherein said input means is activated by a single action selected from the group consisting of pressing, pushing, pulling, positioning, turning, sliding, triggering, sensing and any combination thereof.

13. The system according to claim 1, wherein said input means is activated by a single activation element selected from the group consisting of spring switch, pressing button, pushing button, pulling button, positioning button, turning button, sliding button, triggering button, sensing button and any combination thereof.

14. The system according to claim 1, wherein said system increases the health-care services quality and/or income ratio according to accessibility indicators.

15. The system according to claim 1, wherein said processor is adapted as a communicable device selected from the group consisting of a tablet, a kiosk, a CPU, a smart phone, a telephone and any combination thereof.

16. The system according to claim 1, wherein said system further schedules medical encounters between patient and a physician, using a predetermined rules and subject to instructions by both physician and said patient.

17. The system according to claim 1, wherein said system additionally comprising at least one algorithm selectively executed based on at least a portion of the received information.

18. The system according to claim 17, wherein said algorithm scores at least a portion of the received information and diagnoses a medical condition associated with an executed medical algorithm.

19. A combined device for primary laryngoscopic and otoscopic examinations;
said device comprising:
a. an image sensor;
b. a first lens cooperatively disposed in front of said image sensor such that an image of a larynx of a patient is formed on said image sensor when said image sensor and said lens are inserted into a mouth cavity of said patient;
c. at least one source of light configured for illuminating of said larynx, an acoustic duct and a drum membrane;
d. a speculum configured for translating said light into said acoustic duct;
wherein said device is rearrangeable between a first arrangement for said laryngoscopic examination firand a second arrangement for said otoscopic examination; said first arrangement comprises said image sensor, first lens and at least one source of light cooperatively insertable into said mouth cavity; in said second arrangement, said speculum is disposed before said at least one source of light such that said light is inserted into end butt face and propagates therewithin in a lightguide manner; said speculum is provided with a second lens configured cooperatively with said first lens for forming an image of said acoustic duct and a drum membrane.

20. The combined device according to claim 19, wherein said speculum and second lens are disposable.
